(19) United States
(12) Patent Application Publication
(10) Pub. No.: US 2026/0256736 A1
(43) Pub. Date: Sep. 3, 2026

(54) Composition for Liposomal Delivery of Supplemental Urolithin

(71) Applicant: Spartacus Brands LLC, Boca Raton, FL (US)

(72) Inventors: Jeremy Eisenberg, Boca Raton, FL (US); Stephanie Eisenberg, Boca Raton, FL (US); Augustin Quancard, Boca Raton, FL (US); Gregory Papigny, Boca Raton, FL (US); Gary Bitton, Boca Raton, FL (US)

(21) Appl. No.: 19/654,217

(22) Filed: Apr. 21, 2026

Related U.S. Application Data
(63) Continuation-in-part of application No. 18/166,335, filed on Feb. 8, 2023, now abandoned.

(51) Int. Cl. A61K 31/366; A61K 9/00; A61K 9/127

(52) U.S. Cl. CPC A61K 31/366; A61K 9/0053; A61K 9/127

(57) ABSTRACT

A composition formulated to improve the delivery of supplemental urolithin is provided. More particularly, the composition may be configured to improve the delivery of orally consumed supplemental urolithin. The composition comprises supplemental urolithin and a liposomal blend configured to encapsulate the supplemental urolithin. It is contemplated that encapsulating the supplemental urolithin with the liposomal blend improves the bioavailability of the supplemental urolithin by protecting the supplemental urolithin from degradation caused by the body's digestive system. The liposomal blend may be further operative to increase penetration of the supplemental urolithin in the bloodstream by binding to the user's small intestine, permitting the supplemental urolithin to pass directly into the user's bloodstream, where the supplemental urolithin can be delivered to the user's muscles and other desired regions of the body.

### CROSS-REFERENCE TO RELATED APPLICATIONS

This application is a continuation-in-part of U.S. patent application Ser. No. 18/166,335 filed on Feb. 8, 2023, the entire contents of which are hereby incorporated by reference.

### GOVERNMENT CONTRACT

Not applicable.

### STATEMENT RE. FEDERALLY SPONSORED RESEARCH/DEVELOPMENT

Not applicable.

### COPYRIGHT & TRADEMARK NOTICES

A portion of the disclosure of this patent document may contain material which is subject to copyright protection. This patent document may show and/or describe matter which is or may become trade dress of the owner. The copyright and trade dress owner has no objection to the facsimile reproduction by any one of the patent document or the patent disclosure, as it appears in the Patent and Trademark Office patent files or records, but otherwise reserves all copyrights and trade dress rights whatsoever.

### TECHNICAL FIELD

The disclosed subject matter relates generally to drug delivery and more particularly to targeted drug delivery of ingestible supplemental urolithin using liposomes.

### BACKGROUND

Many are turning to supplements for their touted benefits, such as increased energy, improved appearance of skin, and gut health. Supplements generally seek to provide the body with nutrients that either are not sufficient in a person's diet or their body is incapable of producing in sufficient quantities. For example, one common supplement is urolithin, a nutrient naturally produced by the microbiome in a person's body. Urolithin is produced when polyphenols, a nutrient found in foods and drinks such as pomegranate, strawberries, walnuts, raspberries, tea, and even some oaken barrel-aged wines and spirits. However, the production of urolithin occurs by processing the polyphenols from food and drinks with bacteria in the body's microbiome, and many individuals lack the necessary bacteria to produce urolithin, and even those that can naturally produce urolithin often lack the necessary nutrients to produce a detectable amount of urolithin in the body.

Urolithin provides many beneficial results, including the replacement of damaged mitochondria with healthy mitochondria, increased energy levels, slowing the onset of cognitive diseases, reduced inflammation, and anti-aging, making it a highly desired nutrient in the body. As urolithin is still a relatively new discovery, long-term benefits are still being studied, but is showing beneficial results in cancer prevention and increased life expectancy.

Thus, there is a need to increase urolithin in the body through the use of supplements. However, as is the case with many supplements, supplemental urolithin is lost due to the body's digestive system. Given the recent discovery of urolithin and its benefits, there have been limited proposals for improved delivery of supplemental urolithin. One of these proposals, U.S. Pat. No. 11,066,381 to Nakajima, teaches an aqueous solution containing urolithin and collagen, wherein the collagen acts to stabilize the urolithin. Another proposal, U.S. Pat. No. 10,442,784 to Andreux, et al. teaches a protein-containing nutritional supplement comprising urolithins and protein to address muscle-related conditions. However, Nakajima and Andreux do not teach or suggest a carrier to provide a delivery means to reduce the loss caused by the digestive system.

Indeed, the loss of active ingredients, such as supplemental urolithin, is a problem that plagues all drugs and nutritional supplements. As a result, there are many proposals to increase bioavailability, such as essential oils, as proposed in U.S. Pat. No. 7,716,928 to Benet, and menthol, as proposed by Flashner-Barak in U.S. Patent Pub. No. 2004/0,198,646, and even salts as described in U.S. Patent Pub. No. 2019/0,083,407 to Hanna. These proposals, however, introduce additives that may introduce allergens, affect the stability, or may even counteract the active ingredients.

Thus, although various proposals have been made to address the bioavailability of supplements, none provide an improved method of drug delivery for urolithin. Therefore, there remains a need to increase the bioavailability of urolithin to a user.

### SUMMARY AND OBJECTS OF THE INVENTION

The present disclosure is directed to a composition for increased absorption of supplemental urolithin using liposomal drug delivery. More particularly, the composition comprises supplemental urolithin and a liposomal blend operative to permit targeted delivery of the supplemental urolithin to the user. It is contemplated that the liposomal blend may be operative to improve the bioavailability of the supplemental urolithin by preventing degradation caused by the digestive system and permitting the direct release of the supplemental urolithin into a targeted region.

For purposes of summarizing, certain aspects, advantages, and novel features have been described. It is to be understood that not all such advantages may be achieved in accordance with any one particular embodiment. Thus, the disclosed subject matter may be embodied or carried out in a manner that achieves or optimizes one advantage or group of advantages without achieving all advantages as may be taught or suggested.

In accordance with one embodiment, the supplemental urolithin may be urolithin A. Of course, other forms of supplemental urolithin may be utilized. For example, urolithin A glucuronide, urolithin B, urolithin B glucuronide, urolithin D, or any other form of urolithin known in the art is available.

The liposomal blend may comprise liposomes, including, for example, and without limitation, phospholipids, eggs, or any other form of liposomes. Liposomes will be recognized by a person of ordinary skill in that art as a type of artificial vesicle comprising a hydrophobic membrane of at least one lipid bilayer and an aqueous core. Phospholipids, liposomes, and lipids bilayers are all known in the art, and in the interest of brevity, only a brief description of such components has been provided. In certain embodiments, the liposomal blend may comprise a phospholipid-containing material capable of forming liposomal vesicles when present within defined concentration ranges relative to total formulation weight.

The phospholipids may be any phospholipid known in the art, for example, any of phosphatidylcholine, phosphorylethanolamine, phospholipid, or phosphatidylserine. The phospholipids may form a lipid bilayer similar to that of a cell membrane, as such, the phospholipids in the lipid bilayer may be operative to bind with another cell membrane. For example, the cell membrane may correspond with the lining of the small intestine or any other organ in the body. As such, the phospholipids in the lipid bilayer may be operative to bind with the cell membrane of the small intestine. The phospholipids binding to the cell membrane creates an opening in the cell membrane that corresponds with an opening in the liposome to define a passage. As discussed in more detail below, the passage between the liposome and the cell membrane may permit the supplemental urolithin to pass directly into the user's bloodstream.

In one embodiment, the phospholipids may be plant-based. For example, in an embodiment wherein the phospholipid is phosphatidylcholine, the phosphatidylcholine may be extracted from sunflowers. It is contemplated that by using plant-based phospholipids, the composition may be vegetarian, or even vegan, and thus may be available to those with specific dietary needs. Of course, other forms of phospholipids may be utilized.

The supplemental urolithin may be at least partially mixed in the aqueous core of the liposomes. Thus, the liposomes in the liposomal blend may encapsulate the supplemental urolithin, thus protecting the supplemental urolithin from the digestive system, and reducing the amount of supplemental urolithin lost through digestion. Traditionally, ingested supplements are degraded prior to absorption by gastric juices that are encountered throughout the digestive system. However, liposomes comprise phospholipids that form a barrier around the supplemental urolithin to protect against this degradation, permitting the encapsulated supplemental urolithin to reach a targeted region without the effects of degradation. A person of ordinary skill will recognize that by reducing the amount of supplemental urolithin lost through digestion, more of the ingested supplemental urolithin may be absorbed by the user.

Further, the liposomes may be operative to bind to cell membranes, permitting the supplemental urolithin to diffuse across the cell membrane. For example, and without limitation, the phospholipids may bind to a cell membrane, permitting the encapsulated supplemental urolithin to pass directly through the cell membrane and into the cell body. As previously discussed, the binding of the phospholipids to the cell membrane defines a passage. It is contemplated that this passage may permit the supplemental urolithin to directly enter the cell body, without a loss that occurs due to the cell membrane.

Indeed, a person of ordinary skill in the art will recognize the manners in which liposomes may bind to the cell membrane. It is contemplated that the liposomal blend being operative to bind to the cell membrane may permit the supplemental urolithin to pass directly into the targeted region of the user's body, as such, increasing absorption of the supplemental urolithin. Thus, the liposomal blend may protect the supplemental urolithin from degradation that occurs due to the digestive system.

In yet another embodiment, the liposomal blend may be operative to control the release of the supplemental urolithin. It is contemplated that controlling the release of the supplemental urolithin may alter the period of time in which the supplemental urolithin is effective. Further, the controlled release may permit the supplemental urolithin to have an increased half-life, extending an effective period of the supplemental urolithin.

It is further contemplated that the liposomal blend may be further operative to stabilize the composition and thus extend the shelf-life.

It is contemplated that formulating a composition for increased absorption of supplemental urolithin according to the disclosure and claims provided below may have the following advantages:
a) increased absorption of supplemental urolithin;
b) improves the bioavailability of the supplemental urolithin;
c) reduces waste of ingested urolithin caused by the digestive system;
d) controls the release of supplemental urolithin; and
e) increases the stability of the supplemental urolithin.

Thus, it is an object of the invention to increase the absorption of ingested supplemental urolithin.

It is a further object of this invention to reduce supplemental urolithin waste.

It is yet another object of this invention to increase the shelf-life of supplemental urolithin.

It is yet a further object of this invention to distribute supplemental urolithin to targeted areas of the body.

One or more of the above-disclosed embodiments, in addition to certain alternatives, are provided in further detail below. The disclosed subject matter is not, however, limited to any particular embodiment disclosed.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus.

The terms “couple,” “coupled,” “couples,” “coupling,” and the like should be broadly understood and refer to connecting two or more elements or signals, electrically, mechanically or otherwise. Two or more electrical elements may be electrically coupled, but not mechanically or otherwise coupled; two or more mechanical elements may be mechanically coupled, but not electrically or otherwise coupled; two or more electrical elements may be mechanically coupled, but not electrically or otherwise coupled. Coupling (whether mechanical, electrical, or otherwise) may be for any length of time, e.g., permanent or semi-permanent or only for an instant.

### DETAILED DESCRIPTION

Having summarized various aspects of the present disclosure, reference will now be made in detail to various embodiments of the composition. While the disclosure will be described in connection with these embodiments, there is no intent to limit it to the embodiment or embodiments disclosed herein. Rather, the intent is to cover all alternatives, modifications and equivalents included within the spirit and scope of the disclosure as defined by the appended claims. In accordance with one embodiment, the composition may comprise supplemental urolithin and a liposomal blend. The liposomal blend may comprise liposomes, a chemical structure that will be understood by a person of ordinary skill in the art. In the interest of clarity, non-limiting examples and embodiments of liposomes are discussed, though any form or embodiment of liposomes may be utilized. In certain embodiments, the liposomal blend may include a phospholipid complex comprising one or more phospholipids that form liposomal vesicles when present in proportional ranges relative to the total formulation weight.

In one embodiment, the composition may comprise between about 85 to about 99.99 percent by weight supplemental urolithin and between about 0.01 to about 15 percent by weight liposomal blend. In certain embodiments, liposomal vesicle formation within this range may occur within narrower proportional windows of phospholipid complex relative to total formulation weight.

In another embodiment, the composition may comprise between about 90 to about 98 percent by weight supplemental urolithin and between about 2 to about 10 percent by weight liposomal blend. In certain embodiments within this range, the phospholipid-containing portion of the liposomal blend may be present at about 1% to about 3% by weight of the total formulation.

In yet another embodiment, the composition may comprise about 92.08 percent by weight supplemental urolithin and about 7.92 percent by weight liposomal blend. Of course, the composition may comprise the supplemental urolithin and liposomal blend at any concentration as needed or desired. In a further embodiment, the composition may comprise additional ingredients, including, for example, and without limitation, flavoring, coloring, or even other nutraceuticals.

In certain embodiments, the phospholipid-containing component of the liposomal blend is present at about 1% to about 3% by weight of the total formulation, including active and inactive ingredients. In some embodiments, the phospholipid-containing component is present at about 1% to about 2.5% by weight. In further embodiments, the liposomal blend is present at about 5% by weight of the total formulation, wherein the blend comprises approximately 50% sunflower-derived lecithin, corresponding to about 2.5% lecithin by weight of the total formulation.

In one embodiment, the supplemental urolithin may comprise urolithin A. However, any type of urolithin may be utilized, including, and without limitation, urolithin A glucuronide, urolithin B, urolithin B glucuronide, urolithin D, urolithin M-5, urolithin M-6, urolithin M-7, urolithin C, and urolithin E. In the interest of clarity and brevity, an embodiment for urolithin A is described, however, a person of ordinary skill in the art will appreciate that any form of urolithin may be utilized to carry out the disclosure.

The liposomes in the liposomal blend may be any liposomes known in the art. In general, liposomes comprise at least on lipid bilayer surrounding an aqueous core. The lipid bilayer may be formed from phospholipids, which comprise a hydrophilic head and a hydrophobic tail. Liposomes are generally spherical in shape and will be discussed as such for the sake of brevity, however, liposomes may vary in shape and form, and any shape or form may be utilized. A person of ordinary skill in the art will appreciate that liposomes may contain an active ingredient, such as supplemental urolithin, in the aqueous core or between the hydrophobic tails of the lipid bilayer. While the supplemental urolithin may be contained in either, or even both, of the aforementioned locations, in the interest of brevity, reference, is made to the embodiments wherein the supplemental urolithin is contained in the aqueous core. In certain embodiments, formation of such liposomes is dependent upon the relative proportion of phospholipid-containing material within the total composition.

In certain embodiments, the composition is configured such that the supplemental urolithin is fully incorporated into liposomal vesicles formed from the phospholipid-containing component, such that the composition functions as a fully liposomal delivery system with respect to dispersibility and cellular interaction. In this context, the supplemental urolithin is not present in a free or non-vesicular state to any material extent, but is instead associated with vesicular structures that govern delivery behavior. Accordingly, such compositions may be regarded as fully liposomal in terms of functional performance, notwithstanding the possible presence of minor non-liposomal particle populations detectable by analytical techniques.

As used herein, the phrase “fully liposomal” or “100% liposomal,” when used in reference to a composition, refers to a formulation in which the supplemental urolithin is incorporated into liposomal vesicles such that the composition exhibits liposomal delivery behavior with respect to dispersibility and cellular interaction. This definition does not require the absence of all non-liposomal particle populations, and compositions may include minor non-liposomal aggregates detectable by analytical techniques such as dynamic light scattering while still functioning as a fully liposomal delivery system.

A person of ordinary skill in the art will appreciate that encapsulating the supplemental urolithin in the liposomes may be done in any number of manners, and the subsequent embodiments are provided as non-limiting examples only. Generally, preparing the liposomes will comprise drying lipids, dispersing the lipids in an aqueous media to form the liposomes, and purifying the liposome. The aqueous media may comprise the supplemental urolithin that, when encapsulated, may form the aqueous core. In certain embodiments, dispersion parameters and phospholipid concentration may influence vesicle formation and stability.

In one embodiment, the supplemental urolithin may be encapsulated in the liposomes through a passive loading method. For example, the passive loading method may mechanically disperse the supplemental urolithin through any of a mechanical dispersion method, a solvent dispersion method, and a detergent removal method. Of course, other forms of passive loading may be utilized, and the aforementioned are provided as examples only. In the interest of clarity, one embodiment of mechanical dispersion is provided, though any form of passive encapsulation may be utilized. Mechanical dispersion may utilize a process known in the art as sonication, where the lipids and aqueous media are exposed to high-frequency sound waves. It is contemplated that sonication may be operative to create a more homogenous suspension and may reduce large particles.

In another embodiment, the supplemental urolithin may be encapsulated in the liposomes through active loading. Active loading may occur following the formation of liposomes and may permit the supplemental urolithin to be added at a later time. In some embodiments, active loading may be accomplished using gradient loading, which involves a buffer that uses a PH gradient for loading the supplemental urolithin into the liposome. For example, the aqueous core of the liposome may comprise a low PH and may be suspended in a solution comprising a high PH and the supplemental urolithin. The solution may permeate the liposome to equalize the PH, thus delivering the supplemental urolithin into the aqueous core of the liposome.

In one embodiment, the phospholipids may be lecithin phospholipids. In some such embodiment, the lecithin phospholipids may be extracted from sunflowers. More particularly, the lecithin phospholipids may be extracted from sunflower oil. Of course, phospholipids may be extracted from a large number of plants and animals, such as from eggs, meat, seafood, and soy. As such, any form of phospholipids may be utilized, and the aforementioned are provided as non-limiting examples.

The terms “lecithin,” “phospholipids,” and “phosphatidylcholine” are used interchangeably when referring to phospholipid-containing source materials or components thereof. A person of ordinary skill in the art will appreciate that lecithin may comprise additional substances, such as triglycerides, fatty acids, and carbohydrates, and may be provided as a phospholipid complex comprising a mixture of phospholipid species and non-phospholipid constituents. While phospholipids and phosphatidylcholine may be generally understood to comprise a lower concentration of the additional substances, particularly when provided as purified phospholipid fractions relative to crude lecithin mixtures, for the purposes of this disclosure, unless otherwise provided, references to phospholipids and phosphatidylcholine may include lecithin-derived materials, and “phospholipid complex” refers to a compositional ingredient comprising one or more phospholipids, such as sunflower-derived lecithin, prior to vesicle formation.

In certain embodiments, the phospholipid complex comprises lecithin, such as sunflower-derived lecithin. The phospholipid complex may have a lecithin content of approximately 50% by weight, with the remaining portion comprising one or more additional lipid or non-phospholipid constituents. In some embodiments, the lecithin content of the phospholipid complex is about 45% to about 55% by weight. It is contemplated that providing lecithin content within about 45% to about 55% by weight of the overall composition results in a balanced lipid-to-nonlipid ratio that enables reproductible vesicle formation within the defined concentration window while avoiding aggregation, foaming, and structural instability observed at higher effective phospholipid levels.

It should be noted that liposomes refer to vesicular structures that may be formed from the phospholipid complex when the phospholipid-containing component is present within defined concentration ranges relative to total formulation weight and subjected to dispersion conditions described herein. Not all phospholipid-containing compositions form stable liposomal vesicles, and the presence of a phospholipid complex alone does not imply liposome formation. As described herein, liposomal vesicles are formed only within defined concentration ranges of the phospholipid complex relative to the total formulation, and such vesicles are characterized by nanometer-scale particle sizes and polydisperse distributions.

In certain embodiments, vesicle formation exhibits concentration-dependent behavior. That is, increasing phospholipid concentration may not continuously improve performance. Rather, vesicle formation and dispersibility occur within a defined phospholipid concentration window described herein and relative to total formulation weight. This behavior demonstrates that vesicle formation is not a linear function of phospholipid concentration, and that increasing phospholipid content does not proportionally improve vesicle stability.

For instance, in some embodiments, when the phospholipid-containing component is present below the defined functional window, insufficient lipid association may result in reduced vesicle formation and diminished dispersibility. Under such conditions, the system may exhibit reduced nanometer-scale vesicle populations relative to compositions formulated within the defined window.

Likewise, when the phospholipid-containing component exceeds the defined functional window, excess lipid may promote aggregation, coalescence, and/or surface instability. Under such conditions, particle size distributions shift toward larger aggregate populations, including micron-scale structures, with reduced nanometer-scale vesicle dominance. In some implementations, elevated phospholipid concentrations may further result in foaming during dispersion or mixing, which may interfere with uniform vesicle formation and reduce formulation consistency. Indeed, such foaming may interfere with uniform lipid dispersion and thus contribute to instability of vesicular populations.

Accordingly, in certain embodiments, stable liposomal vesicle populations may be reproducibly formed within the defined concentration window, whereas deviations above or below such window may reduce vesicle stability, dispersibility, or uniformity. That is, the defined concentration window provides a structural and functional balance in which stable, monodisperse liposomal vesicles are reproducibly formed, whereas deviations above or below such window result in reduced vesicle integrity, aggregation, or diminished dispersibility.

In some embodiments, the composition may be ingestible. For example, the composition may be orally consumed. In one embodiment, the composition may be in a powdered form. It is contemplated that the composition may be water soluble such that the powdered composition may be at least partially mixed in water. A person of ordinary skill in the art will recognize that the hydrophilic heads of the liposomes in the liposomal blend may permit the liposomes to bind to the water molecules. As such, the composition may be dissolved in water to create a solution. Of course, other liquids may be utilized, such as juice, coffee, milk, or even other liquids or combinations thereof. It is contemplated that the composition may be dissolved in the same liquids as water-soluble solutes, for example, electrolytes, vitamins, or flavoring.

In another embodiment, the composition may be in a tablet form. In yet another embodiment, the composition may be in capsule form. Of course, the composition may be in another form, such as, and without limitation to, gummies, liquid, soft gels, gel caps, sprays, and bars. A person of ordinary skill in the art will recognize that additional ingredients may be needed or desired according to the desired form of the composition. As such, it is contemplated that in some embodiments, the composition may comprise additional ingredients, such as pill capsules, gelatin, flavoring, or any other ingredient as needed or desired.

In another embodiment, the composition may be topically applied. In yet another embodiment, the composition may be intravenously delivered. Of course, the composition may be in any form capable of being delivered to the body.

In some embodiments, the composition may comprise flavoring. The flavoring may be artificial or natural flavoring and be present at any concentration as needed or desired. Of course, in other embodiments, the composition may be unflavored. In yet another embodiment, the composition may comprise natural or artificial coloring.

In yet another embodiment, the composition may comprise additional nutraceuticals. For example, the composition may comprise supplemental proteins, amino acids, vitamins, antioxidants, minerals, fibers, or any other nutraceutical that may be needed or desired. It is contemplated that the additional nutraceuticals may be present at any concentration in the composition as needed or desired.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

### EXAMPLES OF THE PREFERRED EMBODIMENT OF THE COMPOSITION

In order to more fully teach what the Applicant regards as his invention; the following example is given. It should be understood that the formulation set forth in the Example is not to be construed as limiting the scope of the invention, except so far as they yield a composition having the desired properties and characteristics. More particularly, and by way of example, the following chart illustrates a formulation of the same with percentages given by weight of the composition.

### Example 1

| Ingredient | Percentage by weight |
|---|---|
| Urolithin A | 92.08 |
| Liposomal Blend | 7.92 |
| TOTAL: | 100.00% |
| EXAMPLE 2 | |
| Urolithin A | 43.59 |
| Trans-resveratrol | 12.94 |
| Betaine | 7.91 |
| Anhydrous | |
| (TMG) | |
| CoenzymeQ10 | 4.73 |
| Phospholipid | 3.68 |
| complex | |
| Microcrystalline | 27.15 |
| cellulose (MCC) | |
| TOTAL: | 100% |

### Experimental Results

To evaluate the formation, stability, and functional performance of the disclosed liposomal urolithin compositions, analytical testing was conducted using nuclear magnetic resonance (NMR), dynamic light scattering (DLS), comparative cellular modeling, and electron microscopy. The experiments were designed to evaluate (i) liposomal formation within defined phospholipid concentration ranges, (ii) stability and particle size distribution, and (iii) comparative dispersibility and bioavailability potential relative to non-liposomal formulations.

A representative formulation comprising supplemental urolithin A and a phospholipid complex derived from sunflower lecithin was analyzed by NMR and DLS. NMR quantified phospholipid content at less than 0.35% w/w in the tested formulation. Dynamic light scattering identified two particle size populations. A dominant primary population exhibited a hydrodynamic radius of approximately 336 nm, a polydispersity index (PDI) of approximately 0.25, and represented approximately 70% intensity. This population was interpreted as stable liposomal vesicles. A secondary population exhibited a hydrodynamic radius of approximately 7.55 μm with high polydispersity and reduced stability, representing a minority aggregate fraction.

For the purpose of clarity, stability as used herein refers to maintenance of a dominant nanometer-scale vesicle population without transition to micron-scale aggregate populations under tested conditions.

Notwithstanding the low measured phospholipid level, the formulation consistently formed and maintained a dominant vesicular population within the approximately 300-350 nm range, demonstrating stable liposome formation within the defined formulation parameters.

Comparative testing was performed using active yeast cells as a model organism to evaluate relative dispersibility and cellular interaction of liposomal versus non-liposomal urolithin A formulations prepared under otherwise identical conditions. DLS analysis of a 50/50 blend of liposomal urolithin A and active yeast cells demonstrated a dominant particle population within the 100-1000 nm range, with an average radius of approximately 338 nm and a PDI of approximately 0.62. The system exhibited stable vesicle formation and interaction with cellular surfaces.

By contrast, the non-liposomal urolithin A formulation blended with active yeast cells exhibited multiple non-vesicular particle populations, including large aggregate fractions exceeding 1000 nm and an overall average particle radius of approximately 521 nm. The non-liposomal formulation displayed reduced stability and a predominantly polydisperse character relative to the liposomal system.

Quantitative comparison indicated an approximately 21% improvement in polydispersity index for the liposomal system relative to the non-liposomal formulation and an increase in theoretical bioavailability potential of approximately 1.21× under identical conditions. These findings indicate enhanced dispersibility and vesicular interaction in the liposomal composition.

Additional concentration-dependent testing was conducted to evaluate the effect of phospholipid blend levels on vesicle formation. Formulations containing phospholipid levels exceeding approximately 3-4% of total composition exhibited particle agglomeration and phase instability. Testing of formulations containing sunflower-derived phosphatidylcholine above approximately 4% demonstrated dominant non-liposomal aggregate populations exceeding 1000 nm in size and reduced vesicle integrity. Electron microscopy imaging confirmed irregular macro-aggregate formation at higher phospholipid concentrations.

Formulations within a phospholipid blend range of approximately 1% to approximately 2.5% of total composition consistently produced stable, monodisperse vesicular populations in the approximately 300-350 nm range without excessive agglomeration. Deviations above this range resulted in macro-aggregate formation and reduced liposomal stability, while deviations below this range reduced effective vesicle formation.

These experimental findings demonstrate that liposomal formation, stability, and dispersibility are concentration-dependent and occur reproducibly within a defined phospholipid blend range relative to total formulation weight. The data further demonstrate that compositions formulated within the identified range exhibit improved vesicular stability and dispersibility compared to non-liposomal formulations prepared under otherwise identical conditions. Such structural differences are reflected in measurable particle size distributions and polydispersity values obtained by dynamic light scattering analysis.

### CONCLUSIONS, RAMIFICATIONS, AND SCOPE

While certain embodiments of the invention have been illustrated and described, various modifications are contemplated and can be made without departing from the spirit and scope of the invention. For example, the composition may be in any form, including creams, serums, nasal sprays, and transdermal delivery. Accordingly, it is intended that the invention not be limited, except as by the appended claim(s).

The teachings disclosed herein may be applied to other systems, and may not necessarily be limited to any described herein. The elements and acts of the various embodiments described above can be combined to provide further embodiments. All of the above patents and applications and other references, including any that may be listed in accompanying filing papers, are incorporated herein by reference. Aspects of the invention can be modified, if necessary, to employ the systems, functions and concepts of the various references described above to provide yet further embodiments of the invention.

Particular terminology used when describing certain features or aspects of the invention should not be taken to imply that the terminology is being refined herein to be restricted to any specific characteristics, features, or aspects of the composition for liposomal delivery of supplemental urolithin with which that terminology is associated. In general, the terms used in the following claims should not be constructed to limit the composition for liposomal delivery of supplemental urolithin to the specific embodiments disclosed in the specification unless the above description section explicitly define such terms. Accordingly, the actual scope encompasses not only the disclosed embodiments, but also all equivalent ways of practicing or implementing the disclosed system, method and apparatus. The above description of embodiments of the composition for liposomal delivery of supplemental urolithin is not intended to be exhaustive or limited to the precise form disclosed above or to a particular field of usage.

While specific embodiments of, and examples for, the method, system, and apparatus are described above for illustrative purposes, various equivalent modifications are possible for which those skilled in the relevant art will recognize.

While certain aspects of the method and system disclosed are presented below in particular claim forms, various aspects of the method, system, and apparatus are contemplated in any number of claim forms. Thus, the inventor reserves the right to add additional claims after filing the application to pursue such additional claim forms for other aspects of the composition for liposomal delivery of supplemental urolithin.

What is claimed is:

1. A nutritional composition comprising:
supplemental urolithin; and
from about 1% by weight to about 4% by weight liposomal blend comprising phosphatidylcholine derived from sunflower lecithin,
wherein the liposomal blend forms vesicles around the supplemental urolithin A;
wherein the vesicles have an average particle radius of about 300 nm to about 350 nm;
wherein the supplemental urolithin A is incorporated into the liposomal vesicles such that the composition functions as a fully liposomal delivery system with respect to dispersibility and cellular interaction; and
wherein the liposomal blend is effective to both increase the absorption of ingested supplemental urolithin A and reduce the amount of ingested supplemental urolithin lost through digestion.

2. An ingestible composition comprising:
about 85% to about 99.9% by weight supplemental urolithin; and
a liposomal blend.

3. The composition of claim 2, wherein the liposomal blend comprises phosphatidylcholine.

4. The composition of claim 3, wherein the phosphatidylcholine is extracted from sunflowers.

5. The composition of claim 2, wherein the composition comprises from:
about 0.01% to about 15% by weight liposomal blend.

6. The composition of claim 2, wherein the composition comprises from:
about 2% to about 10% by weight liposomal blend; and
about 90% to about 98% by weight supplemental urolithin.

7. The composition of claim 2, wherein the composition comprises from:
about 7.92% by weight liposomal blend; and
about 92.08% by weight supplemental urolithin.

8. The composition of claim 2, wherein the composition is for oral consumption and the liposomal blend encapsulates the supplemental urolithin to increase the absorption of the supplemental urolithin when ingested.

9. An ingestible composition for increased bioavailability comprising:
from about 99.99% to about 85% by weight supplemental urolithin; and
a liposomal blend;
wherein the supplemental urolithin is incorporated into the liposomal vesicles such that the composition functions as a fully liposomal delivery system with respect to dispersibility and cellular interaction.

10. The composition of claim 9, wherein the liposomal blend comprises phospholipids.

11. The composition of claim 9, wherein the liposomal blend comprises phosphatidylcholine.

12. The composition of claim 11, wherein the phosphatidylcholine is extracted from sunflowers.

13. The composition of claim 9, wherein the composition comprises from:
about 0.05% to about 5% by weight the liposomal blend; and
about 95% to about 99.95% by weight supplemental urolithin.

14. The composition of claim 9, wherein the composition comprises from:
about 0.99% by weight the liposomal blend; and
about 99.01% by weight supplemental urolithin.

15. The composition of claim 9, wherein the liposomal blend encapsulates the supplemental urolithin to increase absorption of the supplemental urolithin.

16. The composition of claim 9, wherein the composition is in a powdered form operative to at least partially dissolve in water.

17. The composition of claim 9, wherein the composition is configured in a form selected from a group consisting of a tablet, capsule, powder, spray, gummy, liquid, soft gel, bar, and gel caps.
